(19) United States
(12) Patent Application Publication
(10) Pub. No.: US 2013/0189372 A1
(43) Pub. Date: Jul. 25, 2013

(54) TOPICAL ANTIBIOTIC FORMULATIONS

(71) Applicants: Perry Antelman, Sharon, MA (US); David Goldsmith, Harmony, RI (US); Shalom Lampert, Maalot (IL)

(72) Inventors: Perry Antelman, Sharon, MA (US); David Goldsmith, Harmony, RI (US); Shalom Lampert, Maalot (IL)

(73) Assignee: AIDANCE SKINCARE & TOPICAL SOLUTIONS LLC, Woonsocket, RI (US)

(21) Appl. No.: 13/577,307

(22) PCT Filed: Feb. 6, 2011
(86) PCT No.: PCT/US11/23843

§ 371 Date: Oct. 31, 2012

Related U.S. Application Data
(60) Provisional application No. 61/302,101, filed on Feb. 6, 2010.

(51) Int. Cl. A61K 47/44; A61K 47/02

(52) U.S. Cl. CPC A61K 47/44; A61K 47/02
USPC 424/618

(57) ABSTRACT

A biocompatible antibiotic formulation suitable for application to skin tissue, the formulation including: (a) a silver(II) oxide; (b) a hydrophilic clay; and (c) a base, said silver(II) oxide and said hydrophilic clay being intimately dispersed within a base.

### CROSS-REFERENCE TO RELATED APPLICATIONS

This application draws priority from U.S. Provisional Patent Application Ser. No. 61/302,101, filed Feb. 6, 2010, which is incorporated by reference for all purposes as if fully set forth herein.

### FIELD AND BACKGROUND OF THE INVENTION

The present invention relates to topical antibiotic formulations.

Chronic wound care is a critical and growing issue in healthcare systems. A chronic wound may be defined as a wound that shows no sign of appreciable healing within 2-3 months. Chronic wounds such as skin ulcers are the most common complication of diabetes, which has been termed a “Silent Epidemic”. Above and beyond their economic burden on healthcare systems, chronic wounds represent a debilitating problem having significant clinical and social ramifications. Chronic wounds may be non-responsive or poorly responsive to various known treatments. Consequently, such chronic wounds may become severely infected, leading to gangrene and amputations.

Various silver derivatives have been used in skin creams. One particularly effective group of silver derivatives is the group of silver oxides. Of the oxides, tetrasilver tetroxide (often represented as Ag₂O₃.Ag₂O or Ag₄O₄) is known to be more effective than Ag₂O.

It was reported by U.S. Pat. No. 6,258,385 to Antelman that
“the effects of the electron transfer involved with respect to the tetroxide, phenomenally, rendered it a more powerful germicide than other silver entities . . . . The oligodynamic properties of these entities may be summarized as follows, which is referred to as the Horsfal series:

Ag₄O₄>Ag(III)>Ag(II)>>>>Ag(I)

We have found that while such electron transfer effects, along with various other effects, may be a cause of the superior anti-microbial efficacy, the very same effects may make the tetrasilver tetroxide particularly reactive with respect to other components in topical formulations. In particular, we have found that the propensity of tetrasilver tetroxide to act as an oxidant may greatly compromise the stability of the formulation. The presence of water may serve to accelerate the degradation processes. Moreover, since tetrasilver tetroxide is typically an extremely minor constituent of such formulations, such undesirable side reactions may actually reduce the anti-microbial availability of the tetrasilver tetroxide and of the formulation as a whole.

While some advances have been made in the treatment of wounds, both chronic and acute, complex and superficial, we believe there is a need for further improvements in formulating stable, efficacious topical antibiotic formulations and medical devices, particularly those containing silver oxides. The subject matter of the present disclosure and claims is aimed at fulfilling this need.

### SUMMARY OF THE INVENTION

According to the teachings of the present invention there is provided an antibiotic formulation suitable for application to skin tissue, the formulation including a mixture including a silver(II) oxide and a hydrophilic clay such as a smectite, the mixture being intimately dispersed within a base.

According to still further features in the described preferred embodiments, the base includes a wax.

According to still further features in the described preferred embodiments, the wax includes a solid wax.

According to still further features in the described preferred embodiments, the wax includes a solid wax and a liquid wax ester.

According to still further features in the described preferred embodiments, the mixture includes a humectant.

According to still further features in the described preferred embodiments, the humectant includes a liquid wax ester having an average carbon number of up to 46, up to 44, or up to 42.

According to still further features in the described preferred embodiments, the liquid wax ester has an average carbon number of at least 34, at least 36, or at least 38.

According to still further features in the described preferred embodiments, the liquid wax ester includes jojoba oil and/or hydrogenated jojoba oil.

According to still further features in the described preferred embodiments, the smectite is selected from at least one of the group consisting of bentonite, montmorillonite, and hectorite.

According to still further features in the described preferred embodiments, the smectite includes bentonite and the base includes jojoba oil.

According to still further features in the described preferred embodiments, the formulation contains at least 3% or at least 5%, by weight, of the humectant or the liquid wax ester.

According to still further features in the described preferred embodiments, the formulation contains, by weight, between 3% and 85%, between 5% and 80%, between 10% and 80%, between 12% and 55%, between 15% and 40%, or between 20% and 35% of the liquid wax ester.

According to still further features in the described preferred embodiments, the silver(II) oxide includes, consists largely of, predominantly of, or substantially of, tetrasilver tetroxide.

According to still further features in the described preferred embodiments, the formulation contains, by weight, at least 0.005%, at least 0.01%, at least 0.03%, at least 0.10%, at least 0.20%, at least 0.30%, or at least 0.50%, of the silver(II) oxide.

According to still further features in the described preferred embodiments, the base includes a beeswax.

According to still further features in the described preferred embodiments, the base includes water.

According to still further features in the described preferred embodiments, the formulation contains, by weight, at least 0.3%, at least 1%, at least 2.5%, or at least 4% of a skin-protecting agent.

According to still further features in the described preferred embodiments, the formulation contains, by weight, at least 0.3%, at least 1%, at least 2.5%, or at least 4% zinc oxide.

According to still further features in the described preferred embodiments, the formulation contains, by weight, less than 15%, less than 12%, or less than 10% of the skin-protecting agent.

According to still further features in the described preferred embodiments, the formulation contains, by weight, less than 15%, less than 12%, or less than 10% of the zinc oxide.

According to still further features in the described preferred embodiments, the formulation contains smectite and silver(II) oxide in a weight ratio of up to 600:1, up to 250:1, up to 100:1, up to 50:1, or up to 25:1.

According to still further features in the described preferred embodiments, this weight ratio is at least 0.2:1, at least 0.5:1, at least 1:1, at least 2:1, at least 5:1, at least 10:1, or at least 20:1.

According to still further features in the described preferred embodiments, the smectite includes, largely includes, predominantly includes, or consists essentially of a smectite selected from at least one of the group consisting of bentonite, montmorillonite, and hectorite.

According to still further features in the described preferred embodiments, the formulation contains, by weight, at least 0.3%, at least 1%, at least 2.5%, or at least 4% zinc oxide.

According to another aspect of the present invention there is provided a solid biocompatible formulation suitable for insertion within chronic and acute wounds of humans and animals, the formulation including a topical antibiotic, a biocompatible humectant, and a biocompatible viscosity-building agent, the humectant and the viscosity-building agent intimately mixed within the formulation, the formulation formulated and adapted whereby the formulation remains a solid over at least an entire temperature range of 20° C. to 35° C., the solid formulation having a storage modulus (G′) and a loss modulus (G″), both measured at 25° C. and within a frequency range of 0.1 Hz to 1.0 Hz, and a complex modulus (G*), defined by:

G*=(G′²+G″²)^{1/2}

the formulation having at least one of the following five rheological properties:

(1) in a torque sweep at a frequency of 1.0 Hz, the complex modulus achieves a plateau or a maximum of at least 4.0×10⁴ Pa, at least 6.0×10⁴ Pa, at least 8.0×10⁴ Pa, or at least 10.0×10⁴ Pa;

(2) in the torque sweep, the complex modulus drops sharply, or begins to exhibit non-linear behavior, at an oscillating stress of at least 800 Pa, at least 900 Pa, at least 1000 Pa, at least 1200 Pa, at least 1500 Pa, or at least 2000 Pa;

within the frequency range, at at least one point:

(3) the storage modulus is at least 1.0×10⁴ Pa, at least 2.0×10⁴ Pa, at least 3.0×10⁴ Pa, at least 4.0×10⁴ Pa, at least 5.0×10⁴ Pa, or at least 6.0×10⁴ Pa;

(4) the loss modulus is at least 0.4×10⁴ Pa, at least 0.5×10⁴ Pa, at least 0.6×10⁴ Pa, at least 0.8×10⁴ Pa, at least 1.0×10⁴ Pa, at least 1.5×10⁴ Pa or at least 2.0×10⁴ Pa;

(5) the complex modulus is at least 1.05×10⁴ Pa, at least 1.05×10⁴ Pa, at least 2×10⁴ Pa, at least 3.0×10⁴ Pa, at least 4.0×10⁴ Pa, or at least 6.0×10⁴ Pa.

According to further features in the described preferred embodiments, the complex modulus achieves a plateau or maximum of at least 4.0×10⁴ Pa, at least 6.0×10⁴ Pa, at least 8.0×10⁴ Pa, or at least 10.0×10⁴ Pa.

According to still further features in the described preferred embodiments, the complex modulus drops sharply, or begins to exhibit non-linear behavior, at an oscillating stress of at least 800 Pa, at least 900 Pa, at least 1000 Pa, at least 1200 Pa, at least 1500 Pa, or at least 2000 Pa.

According to still further features in the described preferred embodiments, at at least one point within the frequency range, the storage modulus is less than 1.2×10⁷ Pa, less than 1.0×10⁷ Pa, less than 8×10⁶ Pa, or less than 7×10⁶ Pa.

According to still further features in the described preferred embodiments, at at least one point within the frequency range, the loss modulus is less than 5×10⁶ Pa, less than 3×10⁶ Pa, less than 2×10⁶ Pa, or less than 1×10⁶ Pa.

According to still further features in the described preferred embodiments, at at least one point within the frequency range, the complex modulus is less than 1.2×10⁷ Pa, less than 1.0×10⁷ Pa, less than 8×10⁶ Pa, or less than 7×10⁶ Pa.

According to still further features in the described preferred embodiments, at at least one point within the frequency range, a ratio of the storage modulus to the loss modulus is at least 1.5:1, at least 2.0:1, at least 2.5:1, at least 3:1, at least 4:1, or at least 5:1, and/or the ratio is less than 12:1, less than 10:1, less than 9:1, or less than 8:1.

According to still further features in the described preferred embodiments, at at least one point within the frequency range, the storage modulus is at least 3.0×10⁴ Pa, at least 4.0×10⁴ Pa, at least 5.0×10⁴ Pa, or at least 6.0×10⁴ Pa, and the loss modulus is at least 0.6×10⁴ Pa, at least 0.8×10⁴ Pa, at least 1.0×10⁴ Pa, at least 1.5×10⁴ Pa, or at least 2.0×10⁴ Pa.

According to still further features in the described preferred embodiments, at at least one point within the frequency range, the storage modulus is at least 5.0×10⁴ Pa, or at least 6.0×10⁴ Pa, and the loss modulus is at least 0.8×10⁴ Pa, at least 1.0×10⁴ Pa, at least 1.5×10⁴ Pa, or at least 2.0×10⁴ Pa.

According to still further features in the described preferred embodiments, a ratio of the storage modulus to the loss modulus is at least 1.5:1, at least 2.0:1, at least 2.5:1, at least 3:1, at least 4:1, or at least 5:1, and/or the ratio is less than 12:1, less than 10:1, less than 9:1, or less than 8:1, substantially throughout the frequency range.

According to still further features in the described preferred embodiments, the storage modulus is at least 3.0×10⁴ Pa, at least 4.0×10⁴ Pa, at least 5.0×10⁴ Pa, or at least 6.0×10⁴ Pa, substantially throughout the frequency range.

According to still further features in the described preferred embodiments, the loss modulus is at least 0.6×10⁴ Pa, at least 0.8×10⁴ Pa, at least 1.0×10⁴ Pa, at least 1.5×10⁴ Pa, or at least 2.0×10⁴ Pa, substantially throughout the frequency range.

According to still further features in the described preferred embodiments, the storage modulus is at least 5.0×10⁴ Pa, or at least 6.0×10⁴ Pa, substantially throughout the frequency range.

According to still further features in the described preferred embodiments, the loss modulus is at least 1.0×10⁴ Pa, at least 1.5×10⁴ Pa, or at least 2.0×10⁴ Pa, substantially throughout the frequency range.

According to still further features in the described preferred embodiments, the water concentration within the formulation is at least 5%, at least 7%, at least 10%, at least 20%, at least 30%, at least 40%, at least 50%, or at least 60%.

According to still further features in the described preferred embodiments, the concentration of the antibiotic within the formulation is at least 0.1%, at least 0.2%, at least 0.4%, at least 0.7%, or at least 1%.

According to still further features in the described preferred embodiments, the antibiotic is present within the formulation in a therapeutically effective concentration for treatment of topical skin infections.

According to still further features in the described preferred embodiments, the antibiotic is selected from the group of topical antibiotics consisting of silver(II) oxide, silver(I) oxide, silver sulfadiazine, Bacitracin, Neomycin, Erythromycin and Chloramphenicol.

According to still further features in the described preferred embodiments, the antibiotic consists largely of, predominantly of, or substantially of, the silver(II) oxide.

According to still further features in the described preferred embodiments, the formulation contains, by weight, at least 0.10%, at least 0.20%, at least 0.30%, or at least 0.50%, silver(I) oxide and/or silver(II) oxide.

According to still further features in the described preferred embodiments, the humectant and the viscosity-building agent are selected, and the formulation is adapted, whereby a melting temperature of the formulation is at least 40° C., at least 45° C., at least 50° C., or at least 75° C.

According to still further features in the described preferred embodiments, the formulation is a putty at 20° C. or at 22° C.

According to still further features in the described preferred embodiments, the formulation is a putty at 35° C. or at 37° C.

According to still further features in the described preferred embodiments, the formulation is a putty throughout the temperature range.

According to still further features in the described preferred embodiments, the formulation contains at least 1%, at least 1.5%, at least 2.5%, at least 3%, at least 4%, at least 7%, at least 12%, at least 20%, or at least 30% of the humectant.

According to still further features in the described preferred embodiments, the formulation contains less than about 55%, less than 50%, less than 48%, less than 45%, or less than 40%, of the humectant.

According to still further features in the described preferred embodiments, the humectant includes, largely includes, predominantly includes, or consists essentially of a liquid wax ester.

According to still further features in the described preferred embodiments, the formulation further includes an absorbefacient.

According to still further features in the described preferred embodiments, the formulation further includes an absorbefacient, wherein a combined weight content of the viscosity-building agent and the absorbefacient within the formulation is at least about 4%, at least 6%, at least 8%, at least 10%, or at least 15%.

According to still further features in the described preferred embodiments, the combined weight content is in a range of about 8% to 70%, about 8% to 65%, or about 10% to 50%.

According to still further features in the described preferred embodiments, the viscosity-building agent includes, largely includes, or consists essentially of at least one of a hydrophilic clay, a flour, and a starch.

According to still further features in the described preferred embodiments, the absorbefacient includes, largely includes, or consists essentially of at least one of a hydrophilic clay, a flour, and a starch.

According to still further features in the described preferred embodiments, the hydrophilic clay is selected from at least one of the group of hydrophilic clays consisting of a smectite, sepiolite, and palygorskite.

According to still further features in the described preferred embodiments, the smectite is selected from at least one of the group consisting of bentonite, montmorillonite and hectorite.

According to still further features in the described preferred embodiments, a weight ratio of the at least one viscosity-building agent and absorbefacient to humectant is at least 0.25:1, at least 0.4:1, at least 0.6:1, at least 1:1, and more typically, about 1.5:1 to 5:1, about 2:1 to 5:1, or about 2:1 to 4:1.

According to still further features in the described preferred embodiments, the humectant includes jojoba oil, hydrogenated jojoba oil.

According to still further features in the described preferred embodiments, the humectant includes, largely includes, or consists essentially of jojoba oil.

According to still further features in the described preferred embodiments, the formulation further includes at least 0.3%, at least 1%, at least 2.5%, or at least 4% of a skin-protecting agent.

According to still further features in the described preferred embodiments, the skin-protecting agent includes zinc oxide.

According to still further features in the described preferred embodiments, the formulation contains, by weight, less than 15%, less than 12%, or less than 10% of the skin-protecting agent.

According to still further features in the described preferred embodiments, the formulation is an elastic, moldable formulation.

According to still further features in the described preferred embodiments, the formulation is adapted whereby a plug or piece of the formulation may be fit or shaped to a contour of a wound cavity.

According to still further features in the described preferred embodiments, the formulation is adapted whereby a plug or piece of the formulation may be inserted into a wound cavity in an integral fashion.

According to still further features in the described preferred embodiments, the formulation is adapted wherein a plug or piece of the formulation securely holds position within a wound cavity.

According to still further features in the described preferred embodiments, the formulation and/or a plug or piece of the formulation is adapted to be removed from a wound cavity in an integral fashion.

According to still further features in the described preferred embodiments, the formulation and/or a plug or piece of the formulation is adapted to provide a gentle pressure against a surface within a wound cavity.

According to still further features in the described preferred embodiments, the formulation and/or a plug or piece of the formulation is adapted to be removed from the wound cavity in an integral fashion, after contacting the surface within the cavity for at least 4 hours, at least 12 hours, or at least 24 hours.

According to another aspect of the present invention there is provided a formulation suitable for application to skin tissue, substantially as described herein, the formulation including any feature described, either individually or in combination with any feature, in any configuration.

According to yet another aspect of the present invention there is provided a wound dressing including any of the formulations described herein.

According to still further features in the described preferred embodiments, the wound dressing includes an adhesive-containing bandage, a cotton roll bandage, or a gelable polymer.

According to yet another aspect of the present invention there is provided a method of producing a composition, formulation, or medical device, the method including any feature described, either individually or in combination with any feature, in any configuration.

According to yet another aspect of the present invention there is provided a method of treating a wound, the method including any feature described, either individually or in combination with any feature, in any configuration.

According to yet another aspect of the present invention there is provided a method of treating a wound, the method including: providing a portion of any one of the formulations disclosed herein; and contacting a surface of the wound with the portion.

According to further features in the described preferred embodiments, the surface is disposed within a cavity of the wound.

According to still further features in the described preferred embodiments, the method further includes the step of: shaping the portion of formulation according to a contour of a cavity of the wound.

According to still further features in the described preferred embodiments, the method further includes the step of: inserting the portion of formulation within the cavity.

According to still further features in the described preferred embodiments, the method further includes the step of: inserting the portion of formulation within the cavity.

According to still further features in the described preferred embodiments, the method further includes the step of: inserting the portion of formulation within the cavity, to effect the contacting of the surface of the wound.

According to still further features in the described preferred embodiments, the method further includes the step of: inserting the portion of formulation within the cavity, whereby the portion exerts a gentle pressure on the surface of the wound.

According to still further features in the described preferred embodiments, the portion of formulation is a single, integral or monolithic portion of formulation.

According to still further features in the described preferred embodiments, the formulation has a substantially non-stick surface with respect to the surface of the wound.

According to still further features in the described preferred embodiments, the contacting of the surface of the wound effects a delivery of the humectant to the surface.

According to still further features in the described preferred embodiments, the contacting of the surface of the wound effects a delivery of the antibiotic to the surface.

According to still further features in the described preferred embodiments, the contacting of the surface of the wound enables an absorbefacient in the portion to withdraw a liquid disposed within the wound.

According to still further features in the described preferred embodiments, the portion contacts the surface of the wound whereby the portion is secured within the surface or within the cavity.

According to still further features in the described preferred embodiments, the portion contacts the surface of the wound for a continuous treatment period of at least 1 hour, at least 4 hours, at least 12 hours, at least 24 hours, at least 48 hours, or at least 72 hours.

According to still further features in the described preferred embodiments, the method further includes the step of: removing the portion of formulation from the cavity, after completing the continuous treatment period.

According to still further features in the described preferred embodiments, the method further includes the step of: removing the portion of formulation from the cavity, in integral or monolithic form, typically after completing the continuous treatment period.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 provides a plot of the storage modulus G′ and the loss modulus G″, as a function of frequency, for a first formulation of the present invention;

FIG. 2 provides a plot of the storage modulus G′ and the loss modulus G″, as a function of frequency, for a second formulation of the present invention;

FIG. 3 shows a torque sweep as a function of the oscillating stress, for a third sample;

FIG. 4 provides a plot of the storage modulus G′ and the loss modulus G″, as a function of frequency, for the third formulation of the present invention;

FIG. 5 provides bar graphs of the zones of inhibition of various formulation of the present invention; and

FIG. 6 provides bar graphs showing clinical wound closure data from comparative clinical trials in which the use of an exemplary putty of the present invention is tested against the use of a silver oxide ointment and against a conventional treatment protocol.

### DESCRIPTION OF THE PREFERRED EMBODIMENTS

Before explaining at least one embodiment of the invention in detail, it is to be understood that the invention is not limited in its application to the details set forth in the following description. The invention may be capable of other embodiments or of being practiced or carried out in various ways. Also, it is to be understood that the phraseology and terminology employed herein is for the purpose of description and should not be regarded as limiting.

We have found that tetrasilver tetroxide may be particularly reactive with respect to other components in topical formulations, thereby compromising the stability of the formulation. In particular, we have found that the carrier base of the formulation may be attacked or broken down by the tetrasilver tetroxide. The formulation may then acquire, disadvantageously, a dark brown or black color.

Moreover, differences in physical properties such as specific gravity, along with differences in chemical properties such as polarity, may cause stratification of the formulation, whereby liquid oil rises to the top of the material, tetrasilver tetroxide and the like sink to the bottom, and hard waxes and the like dominate an intermediate phase disposed therebetween.

One aspect of the present invention relates to a formulation or medical device, based on tetrasilver tetroxide, that may be particularly efficacious in various bacteriostatic or bacteriocidal applications. Such formulations or medical devices may exhibit greatly improved stability, along with efficacy in the inhibition, treatment and cure of various medical conditions, including dermatological conditions. Various formulations of the present invention exhibit no phase separations after two months, after six months, or after over 1 year of shelf life.

With regard to water-based, or water-containing formulations of the present invention, such formulations may include at least 0.25%, at least 0.35%, at least 0.5%, or at least 0.7% of at least one clay such as sepiolite and palygorskite, and/or a smectite clay such as bentonite. The smectite may advantageously be a hydrated smectite. Typically, these formulations may contain less than 20%, less than 12%, less than 10%, less than 8%, or less than 6% of the smectite or clay. The hydrophilic clay or smectite may be an artificial (synthetic or engineered) or processed clay or smectite, in which, by way of example, one or more cationic species may be substituted by one or more other cationic species to achieve a material having specific or pre-determined properties.

The water-based formulations of the present invention may include at least 0.001%, at least 0.01%, at least 0.02%, at least 0.05%, at least 0.15%, or at least 0.3% tetrasilver tetroxide. Typically, these formulations may contain less than 5%, less than 3%, less than 1.5%, or less than 1% tetrasilver tetroxide.

These formulations may advantageously contain one or more liquid wax such as a liquid wax ester. The weight content of liquid wax ester (e.g., including or consisting largely or essentially of jojoba oil) may be at least 3%, or between 5% and 55%, and may depend on the specific application. Typically, the liquid wax ester content may be between 5% and 50%, between 10% and 50%, between 10% and 45%, between 12% and 45%, between 15% and 40%, or between 20% and 35%. The characteristics of the liquid wax ester are provided in greater detail hereinbelow.

The water-based formulations of the present invention may advantageously include zinc oxide, and typically contain at least 0.1%, at least 0.3%, at least 0.5%, or at least 1% zinc oxide. Typically, these formulations may contain less than 8%, less than 5%, or less than 3.5% zinc oxide.

The water content of the water-based formulations of the present invention may be at least 25%, at least 35%, or at least 40%, by weight, and more typically, within a range of 50% to 90%, 55% to 85%, 55% to 80%, or 60% to 75%, by weight, depending on the application.

With regard to oil-based formulations, the oil-based formulations of the present invention may include up to 4% of a clay or smectite clay such as bentonite. The smectite clay may advantageously be a hydrated smectite. Typically, the formulations include at least 0.10%, at least 0.25%, at least 0.35%, or at least 0.45% of the smectite. These formulations may typically contain less than 3%, less than 2.5%, or less than 2% of the smectite.

The oil-based formulations of the present invention may include at least 0.05%, at least 0.10%, at least 0.15%, at least 0.3%, at least 0.5%, or at least 0.65% tetrasilver tetroxide. Typically, these formulations may contain less than 3.5%, less than 2.5%, or less than 2% tetrasilver tetroxide.

These formulations may advantageously contain one or more liquid wax ester, of which jojoba oil may be a presently preferred example. The liquid wax ester content may be at least 3%, at least 5%, at least 10%, at least 20%, or at least 30%, and more typically, between 35% and 95%, by weight. In ointments, the content of liquid wax ester may be between 40% and 95%, between 50% and 90%, between 55% and 85%, or between 60% and 85%, by weight.

The liquid wax ester may advantageously have an average carbon number of up to 46, up to 44, or up to 42.

The liquid wax ester may advantageously have an average carbon number of at least 34, at least 36 or at least 38.

The carrier in these formulations may advantageously contain a solid wax such as beeswax. The content of the solid wax in the formulations may be at least 1%, typically up to 25%, and more typically, between 3% and 20%, by weight. In various ointments, the solid wax content may be between 5% and 20%, between 8% and 20%, between 10% and 18%, or between 10% and 16%, by weight.

The carrier in these formulations may contain petrolatum or the like. The weight content of the petrolatum in the formulations may be at least 3%, at least 5%, at least 10%, at least 25%, at least 40%, and more typically, between 45% and 98%. In formulations containing a solid wax such as beeswax, the content of petrolatum may be adjusted to provide a formulation of the desired consistency.

The oil-based formulations of the present invention may advantageously include zinc oxide, and typically contain, by weight, at least 0.3%, at least 1%, at least 2.5%, or at least 4% zinc oxide. Typically, these formulations may contain less than 15%, less than 12%, or less than 10% zinc oxide.

The oil-based formulations of the present invention may advantageously include various essential oils, including palmarosa oil and/or melissa oil.

With regard to emulsion-based formulations, the emulsion-based formulations of the present invention may include, by weight, up to 8% of a clay or smectite clay such as bentonite. The smectite clay may advantageously be a hydrated smectite clay. Typically, the formulations include at least 0.10%, at least 0.25%, at least 0.35%, or at least 0.45% of the smectite. These formulations may typically contain less than 5%, less than 2.5%, or less than 2% of the smectite.

Another aspect of the present invention relates to a solid or substantially solid formulation or medical device, typically having a putty-like consistency, which may be particularly efficacious as a topical antibiotic in various applications. Such formulations or medical devices may exhibit superior oxidative stability and superior phase stability, along with efficacy in the inhibition, treatment and cure of various dermatological conditions. This formulation may be particularly efficacious in the treatment of bedsores, diabetic ulcers such as diabetic foot ulcers, puncture wounds, and the like.

The inventive putty formulation may include at least one viscosity-building agent, typically including a hydrophilic clay or smectite such as bentonite or hectorite, or an organoclay such as a bentonite or hectorite organoclay, a humectant, typically including an oil or liquid wax ester such as jojoba oil, and a base liquid, typically water or an aqueous solvent. The formulation may advantageously include an absorbefacient.

The viscosity-building agent may include, largely include, predominantly include, or consist essentially of a flour (such as wheat flour, corn flour, and/or rice flour) and/or a starch (such as corn starch or potato starch).

The formulation may advantageously include, in addition to an antibiotic agent, at least one preservative adapted to inhibit bacterial and/or fungal growth within the putty formulation. Preferably, the preservative, or combination of preservatives, should be effective against bacteria, molds and yeasts. Such preservatives may include at least one of benzoic acid, salicylic acid, and various parabens. While various preservatives are known to those of ordinary skill in the art of cosmetic and pharmaceutical formulations, it will be appreciated that the chemical compatibility with silver(II) and silver(I) oxide must be tested, for those formulations containing such silver oxides.

Preferred antibiotics may include at least one silver oxide. Preferably, the inventive solid or substantially solid formulation may include a silver(II) oxide such as tetrasilver tetroxide, or a silver(I) oxide such as Ag2O or silver sulfadiazine. To benefit from the bacteriostatic and antibiotic properties of the silver(II) oxide, the formulation may contain, by weight, at least 0.025% of the silver(II) oxide, and more typically, at least 0.05%, at least 0.10%, at least 0.25%, or 0.25% to 3.5 or 4% thereof. To benefit from the bacteriostatic and bacteriocidal properties of the silver(I) oxide, the formulation may contain, by weight, at least 0.05% of the silver(I) oxide, and more typically, at least 0.10%, at least 0.25%, or 0.25% to 3.5% thereof.

In topical applications such as the treatment of chronic wounds and acute wounds, the inventive formulation preferably exhibits particular mechanical, physical, bacteriocidal, palliative, moisturizing, and skin-protecting or skin-building properties. It is also essential that the various components of the formulation are biocompatible and are compatible with one another.

In some applications, it may be essential for the inventive formulation to be highly absorbefacient, in order to dry up fluid serving as a medium for microbial growth. However, we have found that a delicate balance may exist between inducing absorption and moisturization. Without a suitable moisturization agent or means, the absorption process may disadvantageously dry up the surrounding tissue, which may promote tissue irritation and skin cracking and induce pain, discomfort, and even additional infection. Moreover, we have found that the activity of various antibiotic agents (e.g., silver(II) oxide) may be compromised in dry environments, further constraining the balance between formulation absorption and moisturization.

To this end, we have found that the putty or plaster formulation of the present invention may advantageously include at least about 1%, at least about 1.5%, at least about 2.5%, at least about 3%, at least about 4%, and preferably, about 4% to 55%, about 4% to 50%, about 4% to 45%, about 5% to 40%, about 5% to 30%, or about 5% to 20%, by weight, of a humectant such as a liquid wax ester and/or an oil. The humectant may typically include, largely conclude, or consist mainly or predominantly of, a liquid wax ester such as jojoba oil. Additional humectants will be readily apparent to those of ordinary skill in the art.

The humectant may serve to mitigate or otherwise counter the drying effect of the absorbefacient. At higher concentrations of humectant, the humectant may leak out, ooze out, or be otherwise discharged from the formulation, making the use of the formulation less clean and convenient for medical practitioners and the patient.

Typically, the putty formulation may include at least about 2%, at least about 5%, at least about 8%, at least about 12%, or at least about 20%, by weight, and preferably, about 2% to 50%, about 3% to 45%, or about 4% to 40%, by weight, of at least one such absorbefacient. In these concentrations, the absorbefacient may serve a dual function as a viscosity-building agent. Various phyllosilicates or clays, including smectites, sepiolite and palygorskite, or organoclays such as disteardimonium bentonite may advantageously behave both as an absorbefacient and as a viscosity-building agent. The smectite may include various natural and synthetic forms of bentonite, montmorillonite and hectorite. It may be appreciated by one of skill in the art that hectorite may be somewhat more potent than bentonite and montmorillonite as an absorbefacient and as a viscosity-building agent, on a per-weight basis, such that lower concentrations of hectorite may be used to achieve the desired results. Those of ordinary skill in the art may readily identify other absorbefacient substances that may be suitable for use in the formulations according to the present invention.

It must be emphasized that the inventive formulation may be therapeutically effective in the treatment of wounds and skin infections, even without an antibiotic agent. Without wishing to be bound by theory, the inventors believe that the absorbefacient nature of the formulation is efficacious in reducing the moisture within the wound cavity, negatively impacting the growth environment of the microorganisms.

The inventive putty formulation may further include a skin-protecting or skin-building agent. Typically, the formulation may advantageously include at least 0.2%, and more typically, 1% to 15% or 2% to 10%, by weight, of the skin-protecting or skin-building agent. One presently preferred agent is zinc oxide.

The solvent typically includes water. Water may constitute at least 2%, at least 5%, at least 10%, at least 25%, at least 35%, or at least 40%, by weight, of the inventive formulation, and more typically, about 40 or 45% to 75%, or about 50% to 70% thereof.

We have discovered that with regard to various formulations of the present invention, a high weight ratio of the smectite (or more generally of the total weight of the at least one viscosity-building agent and absorbefacient) to the at least one antibiotic (e.g., Ag2O, a silver(II) oxide such as tetrasilver tetroxide, or Bacitracin, Neomycin and the like) may not reduce the anti-microbial efficacy of the formulation. Weight ratios of up to 600:1 (smectite to antibiotic such as silver(II) oxide), up to 250:1, up to 100:1, up to 50:1, or up to 25:1 may display no decrease in anti-microbial efficacy (relative to substantially identical formulations having no smectite content) with respect to various skin-related microorganisms.

In many formulations of the present invention, the weight ratio of the smectite (or more generally of the total weight of the at least one viscosity-building agent and absorbefacient) to the at least one antibiotic is at least 0.2:1, at least 0.5:1, at least 1:1, at least 2:1, at least 5:1, at least 10:1, at least 20:1, or at least 50:1.

Bentonite, montmorillonite and hectorite are presently preferred smectites.

With particular regard to the putty formulations (including thick, viscous plaster formulations) of the present invention, the putty formulation may have a weight ratio of at least one viscosity-building agent and absorbefacient (e.g., a smectite) to the at least one antibiotic (e.g., silver(II) oxide) of at least 5:1, and more typically, about 5:1 to 200:1, about 5:1 to 75:1, or about 10:1 to 60:1.

In the putty formulation of the present invention, the weight ratio of the at least one viscosity-building agent and absorbefacient to at least one humectant (e.g., jojoba oil) may be at least 0.25:1, at least 0.4:1, at least 0.6:1, at least 1:1, and more typically, about 1.5:1 to 5:1, about 2:1 to 5:1, or about 2:1 to 4:1.

The inventive putty formulation may have various rheological properties that are particularly suited to various topical applications. For example, the putty may have an overall flexibility that is sufficient to enable molding of the putty to conform or largely conform to the shape of various surfaces. For example, a cavity of a wound or bedsore may be filled or partially filled with the inventive putty, whereby the putty conforms to the shape of the cavity. The putty may be inserted into the wound cavity as an integral piece, or as integral pieces. The putty may exhibit sufficient rigidity or stiffness to maintain its position over time (e.g., at least 1 hour, at least 2 hours, at least 4-12 hours, at least 24 hours, at least 48 hours, or at least 72 hours), within such a cavity, without oozing out, falling out, etc. The putty may exhibit sufficient rigidity or stiffness even as the temperature of the putty increases from room temperature to the temperature within the wound of the patient (human or animal).

The inventive putty formulation may advantageously be adapted to retain its integrity within the wound cavity, whereby the putty may be removed as an integral piece after at least 1 hour, at least 2 hours, at least 4 hours, or even after at least 24-72 hours.

The putty formulation may be rheologically adapted to apply a gentle and/or constant pressure against the surrounding tissue. While such pressure contact may promote improved contact between the antibiotic agent and the microorganisms, the contact may, in medical devices and techniques of the prior art, result in sticking of the medical device (e.g., gauze) to the wound surface. Absorbefacients pressure-contacted with a wound surface may excessively dry out the surface. Such effects may adversely affect wound healing, and may subject the patient to discomfort or acute pain. Absorbefacients pressure-contacted with the wound surface may also disintegrate or stick to the wound surface.

By sharp contrast, the inventive formulation may be adapted to remain integral within the wound cavity, to pressure-contact the wound surfaces without sticking thereto, and to be removed with facility from the wound. The formulation may be loaded with sufficient humectant, whereby excessive drying out of the wound surface is avoided, even over several days of continuous presence within the wound cavity.

Various rheological properties of the inventive putty formulation, such as viscosity and/or complex modulus (G*), may be generally maintained between room temperature (about 20-22° C.) and body temperature (about 32-35° C.). This may not be true for various materials or carriers based on petroleum, by way of example. Thus, the formulation components may be selected, and the formulation may be prepared, whereby the melting temperature of the formulation as a whole, is at least 40° C., at least 45° C., at least 50° C., or more typically, at least 75° C.

In characterizing the rheological properties of the present invention, we have found that the inventive formulation may have a large storage modulus (G′) relative to the loss modulus (G″). Using a rotational rheometer such as a TA Instruments G2 rotational rheometer, we have found that the storage modulus, at any point or at substantially every point in the frequency range of 0.1 Hz to 1.0 Hz, may be at least 0.2×10⁴ Pa, at least 0.5×10⁴ Pa, at least 1.0×10⁴ Pa, at least 2×10⁴ Pa, at least 3.0×10⁴ Pa, at least 4.0×10⁴ Pa, at least 6.0×10⁴ Pa, at least 9.0×10⁴ Pa, or at least 12.0×10⁴ Pa. At any point or at substantially every point in this frequency range, the storage modulus may be less than 1.2×10⁷ Pa, less than 1.0×10⁷ Pa, less than 8×10⁶ Pa, or less than 7×10⁶ Pa. More typically, the storage modulus may be within a range of 3.0×10⁴ Pa to 1.0×10⁷ Pa, within a range of 3.5×10⁴ Pa to 9×10⁶ Pa, within a range of 4.0×10⁴ Pa to 7×10⁶ Pa, or within a range of 5.0×10⁴ Pa to 7×10⁶ Pa.

In further characterizing these structural rheological properties, we have found that, at any point or at substantially every point in the frequency range of 0.1 Hz to 1.0 Hz, the loss modulus of the inventive putty formulation may be at least 0.1×10⁴ Pa, at least 0.4×10⁴ Pa, at least 0.5×10⁴ Pa, at least 0.6×10⁴ Pa, at least 0.8×10⁴ Pa, or at least 1.0×10⁴ Pa. At any point or at substantially every point in this frequency range, the loss modulus may be less than 5×10⁶ Pa, less than 3×10⁶ Pa, less than 2×10⁶ Pa, or less than 1×10⁶ Pa.

The ratio of the storage modulus to the loss modulus, at any point or at substantially every point in the frequency range of 0.1 Hz to 1.0 Hz, may be at least 1.0:1, at least 1.5:1, at least 2.0:1, at least 2.5:1, at least 3:1, at least 4:1, or at least 5:1. This ratio may be less than 12:1, less than 10:1, less than 9:1, or less than 8:1. The ratio of the storage modulus to the loss modulus may be in a range of 2.5:1 to 12:1, 3:1 to 10:1, or 4:1 to 9:1. Some formulations of the present invention have a storage modulus to loss modulus ratio of 4.5:1 to 7.5:1, or 5:1 to 7:1.

The complex modulus (G*), which is defined by the equation:

G*=(G′²+G″²)^{1/2}

may be, at any point or at substantially every point in this frequency range, at least 0.3×10⁴ Pa, at least 0.5×10⁴ Pa, at least 0.7×10⁴ Pa, at least 1.0×10⁴ Pa, at least 2×10⁴ Pa, at least 3.0×10⁴ Pa, or at least 4.0×10⁴ Pa, at least 6.0×10⁴ Pa, at least 9.0×10⁴ Pa, at least 12.0×10⁴ Pa, or at least 12.0×10⁴ Pa. At any point or at substantially every point in this frequency range, the complex modulus may be less than 1.2×10⁷ Pa, less than 1.0×10⁷ Pa, less than 8×10⁶ Pa, or less than 7×10⁶ Pa. More typically, the complex modulus may be within a range of 1.0×10⁴ Pa to 1.0×10⁷ Pa, within a range of 2.0×10⁴ Pa to 1.0×10⁷ Pa, within a range of 3.0×10⁴ Pa to 1.0×10⁷ Pa, within a range of 3.5×10⁴ Pa to 9×10⁶ Pa, or within a range of 4.0×10⁴ Pa to 7×10⁶ Pa.

At at least one point within the frequency range, the ratio of the storage modulus to the loss modulus is at least 1.5:1, at least 2.0:1, at least 2.5:1, at least 3:1, at least 4:1, or at least 5:1, and/or the ratio is less than 12:1, less than 10:1, less than 9:1, or less than 8:1.

### EXAMPLES

Reference is now made to the following examples, which together with the above description, illustrate the invention in a non-limiting fashion.

### Example 1

To a container containing water is added at least one viscosity-building agent, typically a smectite (e.g., a bentonite or montmorillonite powder such as Gelwhite H, produced by Southern Clay Products, Inc., Gonzales, Tex.). The mixture is vigorously mixed or homogenized, typically for 0.5 to 2 hours. The oil and/or liquid wax ester (e.g., jojoba oil) may be introduced to the mixture during the mixing (e.g., blending or homogenizing), typically after the viscosity has been built. Mixing may be continued as the antibiotic (e.g., tetrasilver tetroxide) and various optional ingredients (e.g., skin builders) are introduced. Further mixing may ensue, typically for 5-30 minutes. Viscosity-building agents such as flours and starches may be introduced towards the end of the preparation process; a dough hook may advantageously be used for the subsequent mixing.

### Example 2

A putty formulation was prepared according to the procedure provided in Example 1. The putty contained included approximately 66% water, 24% bentonite, 9% jojoba oil, and 0.88% tetrasilver tetroxide.

### Example 3

A putty formulation was prepared according to the procedure provided in Example 1. The putty contained included approximately 65% water, 23% bentonite, 9% jojoba oil, 2% zinc oxide, and 0.88% tetrasilver tetroxide.

### Example 4

A putty formulation was prepared according to the procedure provided in Example 1. The putty contained approximately 53% water, 37% bentonite, 9% jojoba oil, and 0.88% tetrasilver tetroxide.

### Example 5

A putty formulation was prepared according to the procedure provided in Example 1. The putty contained 53% water, 35% bentonite, 9% jojoba oil, 2% zinc oxide, and 0.88% tetrasilver tetroxide.

### Example 6

A putty formulation was prepared according to the procedure provided in Example 1. The putty contained approximately 41% water, 3.4% bentonite, 14.2% jojoba oil, 41% flour, and 0.5% tetrasilver tetroxide. The putty was highly pliable and exhibited excellent phase stability.

### Example 7

A putty formulation was prepared according to the procedure provided in Example 1. The putty contained approximately 47% water, 10% bentonite, 43% jojoba oil, and under 0.1% tetrasilver tetroxide. The putty formulation was designated as Sample 11010-1.

### Example 8

A putty formulation was prepared according to the procedure provided in Example 1. The putty contained approximately 46% water, 34% bentonite, 13% jojoba oil, 5% zinc oxide, and 1% tetrasilver tetroxide. The putty formulation was designated as Sample 11010-2.

### Examples 9-10

The putty formulations of Example 7 and Example 8 were subjected to rheological evaluation using a TA Instruments G2 rotational rheometer.

Small amplitude oscillatory rheometry was conducted on the provided samples, using a two-centimeter, stainless steel parallel plate geometry. To overcome sample-loading issues, the two samples were placed on the Peltier plate of the rheometer, and partially flatted with a flat Teflon® plate. Two one-millimeter shims were placed on either side of the sample, and a doctor blade was used to trim the sample to approximately 1000 micrometers. The two-centimeter parallel plate was then lowered onto the sample, achieving a gap distance between 1000 and 1050 micrometers.

The samples were initially subjected to a stress sweep at 1 Hz to identify the suitable oscillating torque for the frequency sweep based on waveform shape and the onset of non-linear viscoelastic behavior. Based on this work, a torque of 1000 mN-m was used for 11010-1, and 8000 mN-m for 11010-2.

Frequency sweeps were then conducted on both samples from 0.01 to 100 Hz at 25° C. and the indicated oscillating torques. Ten points were collected per decade of oscillating frequency. The run for sample 11010-1 showed inertial effects at frequencies above 16 Hz; as such, the data curve was truncated.

Both samples exhibit strongly elastic behavior in the regime tested, indicated by a large storage modulus G′ relative to the loss modulus G″. Sample 11010-1 is substantially less stiff than 11010-2 by approximately a factor of 60-70. At 1 Hz, sample 11010-1 had a storage modulus of 6.26×10⁴ Pa, while sample 11010-2 had a storage modulus of 4.32×10⁶ Pa. Both samples show a modest onset of a terminal zone at a frequency of approximately 0.03 Hz, followed by a quasi-plateau modulus. No strain hardening was observed in the achievable upper range of frequencies tested.

The complex modulus, G*, is the resultant vector of the storage and loss modulus. A higher complex or overall modulus indicates a stiffer material, requiring more force to deform the material a set amount. A material with a higher storage modulus relative to the loss modulus is more elastic and will therefore recover more than a material with a closer ratio; the ratio of the loss (G′) to storage (G′) modulus is reported as tan d. A purely elastic material would have a tan d=0, while a purely viscous material would have tan d=∞. The comparison of these parameters at two frequencies is shown in Table 1.

Consistent with the discussion above, there is a large difference in modulus between samples 11010-1 and 11010-2, but only a modest frequency dependence in either sample. Also, the tan d values are quite close between the two samples, indicating a similar relative level of elasticity, albeit requiring different levels of force to achieve the same degree of deformation. Both samples show a modest decrease in tan d, indicating both materials become slightly more elastic with increasing frequency. It should be noted that this test subjects the sample to small amplitudes of deformation; larger degrees of deformation could require different levels of force, hence resulting in a different modulus, but the test implicitly assumes that the experiment is performed in the linear viscoelastic regime of the material.

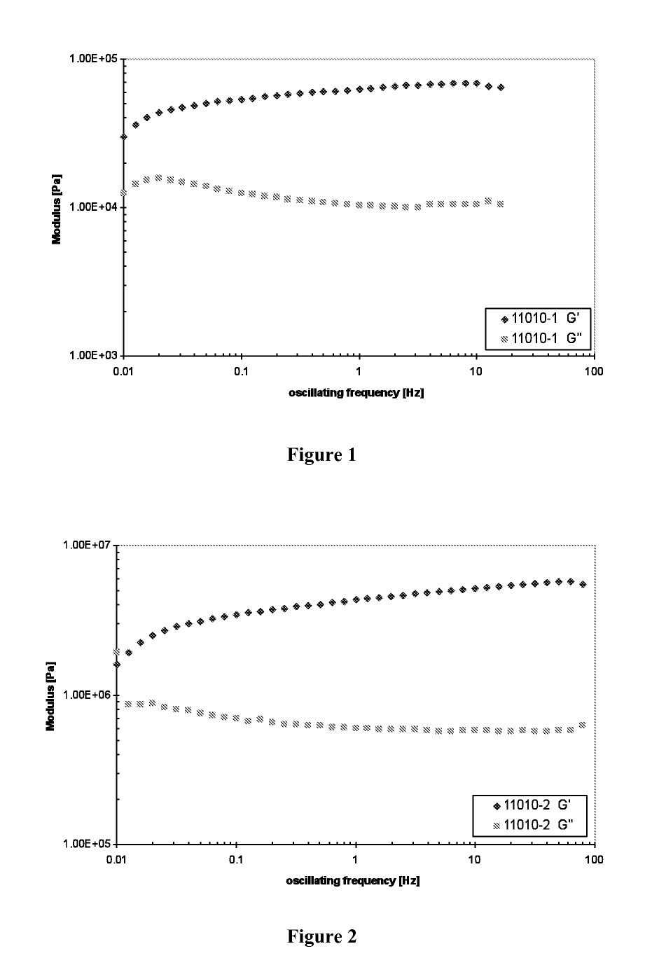

With regard to samples 11010-1 and 11010-2, the storage modulus G′ and the loss modulus G″ are plotted in FIG. 1 and FIG. 2, respectively, as a function of frequency (“Frequency Sweep”). The values of G′, G″ and G* at 0.1 Hz and at 1.0 Hz are provided in Table 1 hereinbelow.

TABLE 1

| | Frequency | | | | |
|---|---|---|---|---|---|
| Sample | [Hz] | G′ [Pa] | G″ [Pa] | G* [Pa] | tan delta (δ) |
| 11010-1 | 0.1 | 5.37E+04 | 1.25E+04 | 5.51E+04 | 0.23 |
| | 1.0 | 6.26E+04 | 1.03E+04 | 6.34E+04 | 0.17 |
| 11010-2 | 0.1 | 3.45E+06 | 6.92E+05 | 3.52E+06 | 0.20 |
| | 1.0 | 4.32E+06 | 5.99E+05 | 4.36E+06 | 0.14 |

### Example 11

A putty formulation was prepared according to the procedure provided in Example 1. The putty contained 42.5% water, 3.3% bentonite, 13.8% jojoba oil, 39.9% flour, and 0.5% tetrasilver tetroxide. The putty was soft and slightly sticky, relative to the formulation of Example 6, but exhibited both high pliability and excellent phase stability.

### Example 12

The putty formulation of Example 11 was subjected to rheological evaluation using a TA Instruments ARG2 rheometer.

Small amplitude oscillatory rheometry was conducted on the provided samples using a TA Instruments ARG2 rheometer. A two-centimeter, stainless steel parallel plate geometry was used to prevent the bridging effects that can occur in cone geometries when particle sizes might be significant. A gap of 1000 microns was used.

The samples were initially subjected to a torque sweep at 1 Hz to identify the suitable oscillating torque for the frequency sweep based on waveform shape and the onset of non-linear viscoelastic behavior. Based on this work, a torque of 1000 μN-m was determined.

A frequency sweep was then conducted on the sample, from 0.01 to 100 Hz at 25° C., using the oscillating torque amplitudes described above. Ten points were collected per decade of frequency.

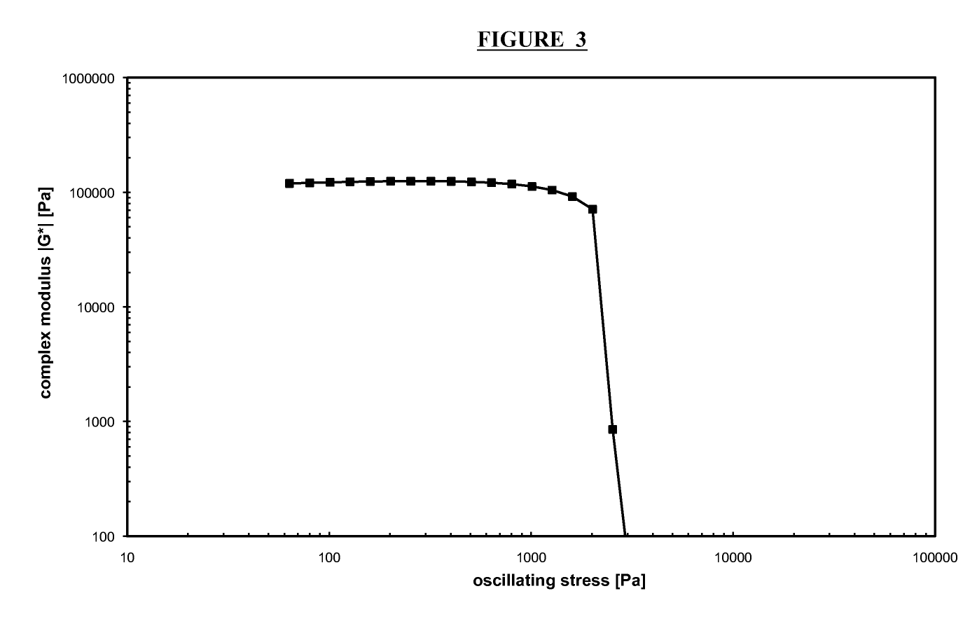

The results from the torque sweep are shown in FIG. 3. The sample showed non-linear behavior at approximately 2,000 Pa.

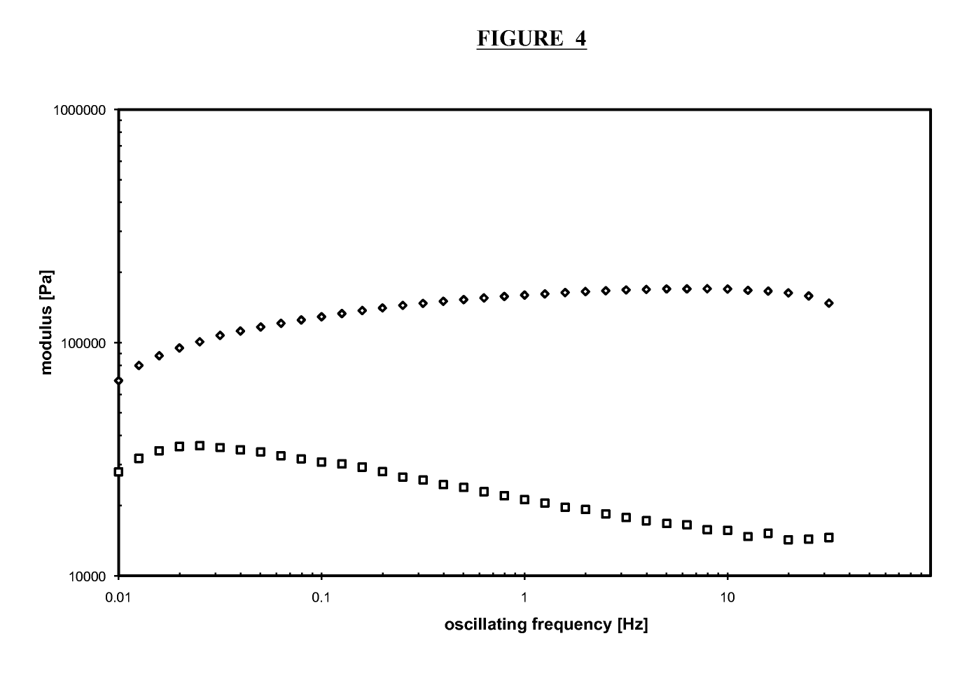

The frequency sweep data are plotted in FIG. 4. The sample exhibited a fairly flat modulus behavior, indicating little dependency on frequency.

This data is summarized at 3 frequencies (0.1 Hz, 1.0 Hz, and 10 Hz) in Table 2. The behavior of the sample was predominantly elastic, with the complex modulus ranging from about 1×10⁵ to 2×10⁵ Pa.

TABLE 2

| | Frequency | | | | |
|---|---|---|---|---|---|
| Sample | [Hz] | G′ [Pa] | G″ [Pa] | G* [Pa] | tan delta (δ) |
| 11264-2 | 0.1 | 1.29E+05 | 3.08E+04 | 1.33E+05 | 0.24 |
| | 1.0 | 1.60E+05 | 2.12E+04 | 1.61E+05 | 0.13 |
| | 10 | 1.70E+05 | 1.57E+04 | 1.71E+05 | 0.09 |

### Example 13

A formulation was prepared according to the procedure provided in Example 1, containing 69.8% water, 9.3% bentonite, 8.1% jojoba oil, 9.3% flour, 3% zinc oxide, and 0.5% tetrasilver tetroxide. The formulation was soft, having a soft putty or plaster-like consistency, and exhibited both high pliability and excellent phase stability.

### Example 14

A formulation was prepared according to the procedure provided in Example 1, containing 68.5% water, 18.3% bentonite, 7.9% jojoba oil, 4.8% zinc oxide, and 0.5% tetrasilver tetroxide. The formulation was soft, having a plaster-like consistency and exhibited both high pliability and excellent phase stability.

### Example 15

The formulation of Example 13 was prepared according to the general procedure provided in Example 1, however, the mixing of the bentonite into the water was conducted for about 10 minutes. Despite having a composition substantially identical to that of Example 13, the formulation failed to develop the requisite viscosity or body. The formulation had a paste-like consistency, even after additional mixing time was provided after the silver oxide and zinc oxide were introduced.

### Example 16

A formulation was prepared according to the procedure provided in Example 1, containing 65.4% water, 11.3% bentonite, 17.8% jojoba oil, 5% zinc oxide, and 0.5% tetrasilver tetroxide. The formulation was soft, exhibiting a plaster-like consistency and exhibited both high pliability and excellent phase stability.

### Example 17

A putty formulation was prepared according to the procedure provided in Example 1, containing 40.6% water, 3.4% bentonite, 14.2% jojoba oil, 40.5% wheat flour, 0.5% tetrasilver tetroxide, 0.5% Allantoin, 0.1% Benzathonium Cl, and 0.5% Lidocaine.

### Example 18

A putty formulation was prepared according to the procedure provided in Example 1, containing 39.0% water, 3.1% bentonite, 13.5% jojoba oil, 40.0% wheat flour, 0.5% tetrasilver tetroxide, 1.0% Clotrimazole, 5% salicyclic acid, and 0.1% colloidal oatmeal.

### Example 19

The anti-microbial efficacy of various formulations was tested and compared using a Kirby-Bauer type test, as follows:

Ready-made Muller-Hilton agar was streaked with the bacterial inoculum using a sterile applicator. The sample was allowed to sit for 5 minutes to ensure that the bacteria adhere to the surface of the agar. Subsequently, an antibiotic sterile blank disc was pressed against a known quantity of the formulation being tested. Multiple duplicate discs were used to verify the data. The disc was pressed against the surface of the agar, making sure not to damage the disc or the agar. Each agar plate was then inverted and allowed to sit in the incubator at 37° C. for 24 hours. The plates were subsequently removed from the incubator, and the zone of inhibition was measured using a ruler.

The anti-microbial efficacy of eight formulations was tested and compared using the procedure detailed above, using Enterococcus faecalis.

Formulation Nos. 1-7 correspond to the formulations produced in Example Nos. 6, 11, 13, 14, 16, 17, and 18. Formulation No. 8 was a control formulation, produced according to the procedure outlined in Example 1. The control formulation was a putty containing: 40% water, 3.4% bentonite, 14.2% jojoba oil, and 40% wheat flour. No antibiotic was included in the control formulation.

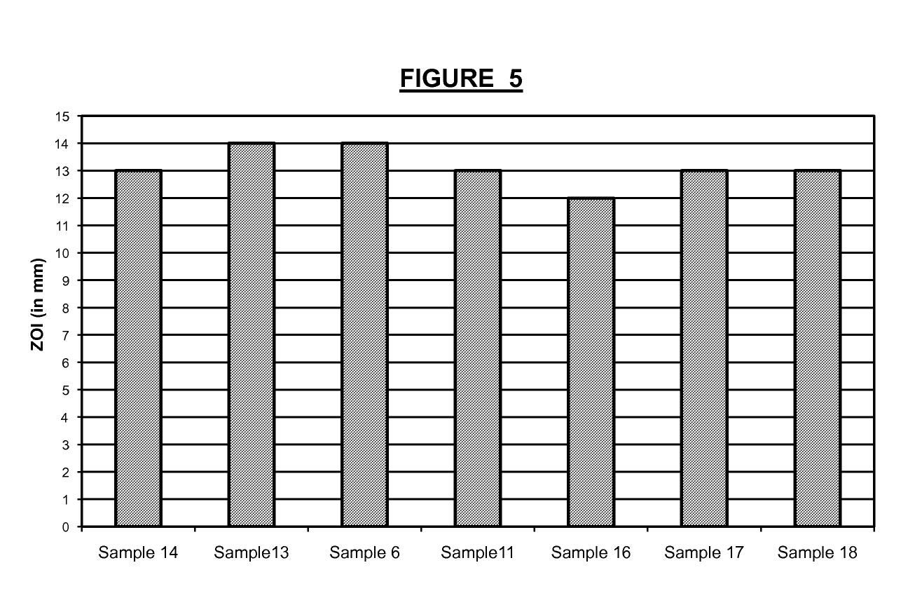

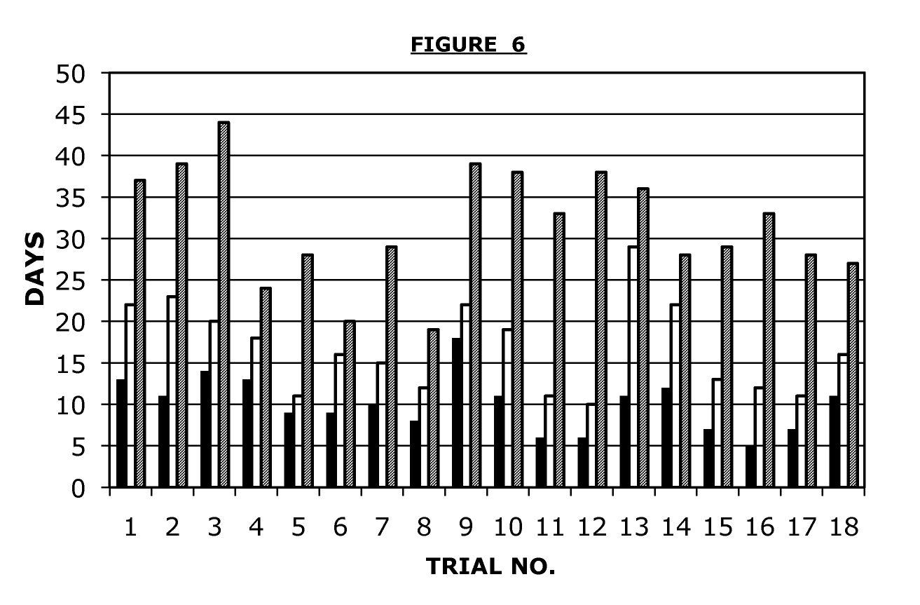

The zone of inhibition for the control formulation was substantially 0 mm. By sharp contrast, the zone of inhibitions for Formulation Nos. 1-7 all fell within a narrow range of about 12-14 mm (see FIG. 5). Such a large zone of inhibition may be considered a clear manifestation of the appreciable antibiotic activity of the inventive formulations, and was obtained using a low concentration of the silver oxide. Moreover, the large zone of inhibition may be especially noteworthy in view of the extremely high viscosities exhibited by the inventive putty formulations.

### Examples 20-26

Formulations having compositions generally along the lines of Example 11, were prepared according to the procedure provided in Example 1. In Example 20, the putty contained 42.5% water, 3.3% bentonite, 13.8% jojoba oil, 39.9% flour, and 0.5% tetrasilver tetroxide, as in Example 11. The flour was a whole wheat flour. The putty was soft and slightly sticky, relative to the formulation of Example 6, but exhibited both high pliability and moldability, and excellent phase stability.

In Example 21, rice bran flour replaced the wheat flour, and the silver(II) oxide concentration was increased to 4%. To obtain a similar consistency as that obtained in Example 20, the ratio of filler (rice flour) to water was increased from 0.94 to 1.48, representing a 58% increase in filler, relative to the wheat flour of Example 20. The putty had a dark gray color, which may largely be due to the relatively high concentration of the silver(II) oxide. The putty was soft and slightly sticky, relative to the formulation of Example 6, and exhibited excellent phase stability. The formulation had a somewhat grainy appearance and was moldable, though less so than the putty of Example 20.

In Example 22, corn starch replaced the wheat flour of Example 20, while the silver(II) oxide concentration was maintained at 0.5%. To obtain a similar consistency as that obtained in Example 20, the ratio of filler (corn starch) to water was increased from 0.94 to 1.09, representing a 17% increase in filler, relative to the wheat flour of Example 20. The putty had a substantially white appearance. The putty was soft and slightly sticky, relative to the formulation of Example 6, and exhibited excellent phase stability. The formulation was moldable, though less so than the putty of Example 20.

In Example 23, potato starch replaced the wheat flour of Example 20, while the silver(II) oxide concentration was increased at 1.5%. To obtain a similar consistency as that obtained in Example 20, the ratio of filler (potato starch) to water was increased from 0.94 to 1.25, representing a 33% increase in filler, relative to the wheat flour of Example 20. The putty had a yellow tinge. The putty was soft and slightly sticky, relative to the formulation of Example 6, and exhibited excellent phase stability. The formulation was more grainy than the putty of Example 22 and was moldable, though less so than the putty of Example 20.

In Example 24, the whole wheat flour of Example 20 was used, but the silver(II) oxide was replaced by silver(I) oxide (0.5%). The appearance of the putty, consistency, and phase stability appeared to be identical, or substantially identical to those of the putty of Example 20.

In Examples 25 and 26, the putty formulation of Example 20 was prepared, again, according to the general procedure of Example 1. In each formulation, a different topical antibiotic material was used instead of the silver(II) oxide. The concentration of each antibiotic material was selected according to the concentration of the antibiotic material in commercially available ointments. Thus, in Example 25, the antibiotic material was clotrimazole, 1% by weight; in Example 26, the antibiotic material was erythromycin, 2% by weight. The appearance, consistency, and phase stability of the putties of Example 25 appeared to be identical, or substantially identical to those of the putty of Example 20.

### Example 27

Fifty four patients were treated at Irvine3 Circulation/Vascular Labs (Chieti-Pescara University, Pescara, Italy). Patients were matched with other patients of similar age and similar general health condition, and having complex ulcers of similar type, size and severity. Effectively, 18 groups of three patients were formed for the purpose of comparative testing.

Within each group of three, a first patient was treated with conventional cleaning and compression management methods. A second patient within each group was treated with an ointment, containing approximately 0.9% silver(II) oxide and 6.8% ZnO in a beeswax and jojoba oil base. The ointment was applied around and at the edge of the ulcerated areas and on the ulceration, following the identical conventional cleaning methods used on the first patient.

The third patient within each group was treated with an antibiotic-containing putty of the present invention. The antibiotic, consisting essentially of tetrasilver tetroxide (silver(II) oxide), was dispersed within the putty, which had the composition of the putty described in Example 6, and was prepared according to the procedure provided in Example 1.

FIG. 6 provides bar graphs showing the wound closure data from each of the 18 comparative clinical trials. In FIG. 6, associated with each trial number are three juxtaposed bar graphs, the right-most of which represents the patient subjected to the conventional treatment, the middle bar graph represents the patient treated with the ointment containing the silver(II) oxide, and the left-most of which represents the patient treated using the formulation of the present invention.

On average, the complex ulcers treated by conventional means required over 31 days to close, on average. The complex ulcers treated with the silver(II) oxide based ointment required almost 17 days to close, on average, an appreciable improvement over the results for the control group. The complex ulcers treated with the antibiotic-containing putty of the present invention closed after just over 10.1 days, on average, about ⅓ of the time required for the wounds of the control group, and about 40% less time with respect to the excellent result achieved using the ointment. The performance of the inventive antibiotic putty is more surprising in view of the relatively low concentration of antibiotic (0.5% silver(II) oxide) in the putty, with respect to the concentration of the same antibiotic (˜0.9% silver(II) oxide) in the antibiotic ointment formulation. The improved performance is even more surprising in view of past experience showing that for a given concentration of antibiotic material, more viscous formulations may considerably less efficacious from a bacteriocidal standpoint.

### Example 28

An exemplary general procedure for producing oil-based tetrasilver tetroxide compositions and formulations according to the present invention is as follows: an oil and/or liquid wax ester such as jojoba oil is heated, preferably to around 80 C. A wax such as beeswax is preferably melted into the oil or liquid wax ester. The material may be mixed thoroughly as it is cooled, typically below about 60 C. Optionally, an essential oil such as palmarosa oil may be added. Mixing may be continued as the tetrasilver tetroxide is introduced, and further mixing may ensue, typically for 0.5 to 2 hours, during cooling of the mixture to below about 40 C. A smectite (such as bentonite) and/or a skin-protecting or skin-building agent such as zinc oxide may be introduced along with the tetrasilver tetroxide, or sometime therebefore or thereafter. The formulation may then be poured into storage containers.

Typically, the formulations contain a total tetrasilver tetroxide content of 0.05% to 3%, by weight, and more typically, 0.1% to 3% tetrasilver tetroxide.

### Example 29

An exemplary general procedure for producing water-based tetrasilver tetroxide compositions and formulations according to the present invention is as follows: to a container containing water is added a viscosity-building agent, typically a smectite (e.g., a bentonite or montmorillonite powder such as Gelwhite H, produced by Southern Clay Products, Inc., Gonzales, Tex.). Other viscosity-building clays, particularly clays in which the silicate layers are disposed in a sandwiched structure, may also be used.

The mixture is mixed or homogenized, typically for 0.5 to 2 hours. Tetrasilver tetroxide may be introduced at this stage of the processing. Optionally, a skin-protecting or skin-building agent such as zinc oxide may be introduced to the mixture, typically along with the tetrasilver tetroxide, or sometime therebefore or thereafter. The oil and/or liquid wax ester (e.g., jojoba oil) may be introduced to the mixture during the mixing (e.g., blending or homogenizing).

Mixing may be continued as the tetrasilver tetroxide is introduced, and further mixing may ensue, typically for 5-30 minutes. The formulation may then be poured into storage containers.

### Example 30

An exemplary general procedure for producing emulsion-based tetrasilver tetroxide compositions and formulations according to the present invention is as follows: a liquid such as water may be mixed or blended, preferably at a high speed. A viscosity-building agent such as a clay (e.g., a smectite such as bentonite or other clays in which the silicate layers are disposed in a sandwiched structure) is preferably mixed into the water. Mixing may be continued, typically for 5 to 120 minutes, to assure a homogenous mixture. Mixing may be continued as an oil and/or liquid wax ester (e.g., jojoba oil) is mixed into the mixture. Mixing may be continued, typically for another 5 to 45 minutes, to assure a substantially homogenous suspension including an emulsion of oil and water, and suspended solids. Optionally, an essential oil such as palmarosa oil may be added. Mixing may be continued as the tetrasilver tetroxide is introduced. The optional introduction of a skin-protecting or skin-building agent such as zinc oxide may be effected along with the introduction tetrasilver tetroxide, or some time therebefore or thereafter, and further mixing may ensue, typically for 1 to 10 minutes. The temperature of the mixture is usually below 50 C. The formulation may then be poured into storage containers.

### Example 31

An oil-based topical tetrasilver tetroxide formulation was prepared according to the procedure provided in Example 28. The ointment produced contained 0.9% bentonite, 12.2% beeswax, 77% jojoba oil, 8.2% zinc oxide, 1% silver(II) oxide, and minute quantities of various essential oils.

### Example 32

A water-based topical tetrasilver tetroxide formulation was prepared according to the procedure provided in Example 29. The water-based cream contained 4.2% bentonite, 28.2% jojoba oil, approximately 0.2% silver(II) oxide, and just over 67% water.

### Example 33

A water-based topical tetrasilver tetroxide formulation was prepared according to the procedure provided in Example 29. The water-based cream contained 2.9% bentonite, 29.8% jojoba oil, 0.45% silver(II) oxide, 2.8% zinc oxide, and about 64% water.

### Example 34

An emulsion-based topical tetrasilver tetroxide formulation was prepared according to the procedure provided in Example 30. The emulsion-based cream contained 600 grams water, 50 grams bentonite, 281 grams jojoba oil, and 0.9 grams tetrasilver tetroxide.

### Example 35

An emulsion-based topical tetrasilver tetroxide formulation was prepared according to the procedure provided in Example 30. The emulsion-based cream contained 600 grams water, 46 grams bentonite, 262 grams jojoba oil, and 0.6 grams tetrasilver tetroxide.

As used herein in the specification and in the claims section that follows, the term “percent”, or “%”, refers to percent by weight, unless specifically indicated otherwise.

Similarly, the term “ratio”, as used herein in the specification and in the claims section that follows, refers to a weight ratio, unless specifically indicated otherwise.

As used herein in the specification and in the claims section that follows, the term “silver (II) oxide” refers to a silver oxide whose unit structure contains silver and oxygen in a substantially 1:1 molar ratio. The term “silver (II) oxide” is specifically meant to include Ag₄O₄ (often represented as Ag₂O₃.Ag₂O) and AgO.

As used herein in the specification and in the claims section that follows, the term “silver (I) oxide” refers to a silver oxide whose unit structure contains silver and oxygen in a substantially 2:1 molar ratio. The term “silver (I) oxide” is specifically meant to include Ag₂O.

As used herein in the specification and in the claims section that follows, the term “antibiotic” refers to a substance that selectively attacks and destroys at least one species or type of microorganism, while exhibiting relative inertness with respect to human and/or mammalian cells. More typically the antibiotic substance selectively attacks and destroys at least one species or type of microorganism that commonly populates the skin, surface wounds, bedsores and the like, while exhibiting relative inertness, with respect to skin cells of humans and/or mammals. The term “antibiotic” is specifically meant to exclude anti-microbial preservatives, both anti-fungal preservatives and anti-bacterial preservatives. Such anti-fungal preservatives include, but are not limited to, compounds such as benzoic and ascorbic acids and salts thereof, and phenolic compounds such as methyl, ethyl, propyl and butyl p-hydroxybenzoate (parabens). Antibacterial preservatives include, but are not limited to, compounds such as quaternary ammonium salts, alcohols, phenols, mercurials and biguanidines. The term “antibiotic” is specifically meant to exclude anti-microbial preservatives such as table salt and the like, vinegar, sodium nitrate, sodium nitrite, and sulfites. The term “antibiotic” is specifically meant to include, without being limited to, silver oxides such as silver(I) oxide and silver(II) oxide, silver sulfadiazine, and any other topical antibiotics that are efficacious in the treatment of serious skin wounds such as bedsores, skin ulcers, and puncture wounds, or that are efficacious in the treatment of mundane skin wounds. The term “antibiotic” is specifically meant to include “classic” topical antibiotics such as Bacitracin, Neomycin, Erythromycin and Chloramphenicol. Additional topical antibiotic substances may be readily apparent to those of ordinary skill in the art.

As used herein in the specification and in the claims section that follows, the term “therapeutically effective amount”, with respect to an antibiotic substance or formulation, refers to a quantity that produces a positive result in the treatment of at least one topical infection.

As used herein in the specification and in the claims section that follows, the term “therapeutically effective concentration”, with respect to an antibiotic substance within a formulation or medical device, refers to a concentration of the antibiotic, within the formulation or medical device, which produces a positive result in the treatment of at least one topical infection.

As used herein in the specification and in the claims section that follows, the term “putty”, with respect to a substance or formulation, is meant to refer solely to the physical consistency of the substance or formulation.

As used herein in the specification and in the claims section that follows, the term “plaster”, with respect to a substance or formulation, is meant to refer solely to the physical consistency of the substance or formulation.

As used herein in the specification and in the claims section that follows, the term “largely includes”, and the like, with respect to a component within a formulation, refers to a content of at least 30%, by weight.

As used herein in the specification and in the claims section that follows, the term “mainly includes”, and the like, with respect to a component within a formulation, refers to a content of at least 50%, by weight.

As used herein in the specification and in the claims section that follows, the term “predominantly includes”, and the like, with respect to a component within a formulation, refers to a content of at least 65%, by weight.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1-71. (canceled)

72. An antibiotic formulation suitable for application to skin tissue, the formulation comprising:
(a) a silver(II) oxide;
(b) a hydrophilic clay; and
(c) a base,
said silver(II) oxide and said hydrophilic clay being intimately dispersed within said base.

73. The formulation of claim 1, said base including a wax.

74. The formulation of claim 2, said wax including a solid wax.

75. The formulation of claim 2, said wax including a solid wax and a liquid wax ester.

76. The formulation of claim 2, further comprising a humectant.

77. The formulation of claim 76, said humectant including a liquid wax ester having an average carbon number of up to 46.

78. The formulation of claim 77, said liquid wax ester having an average carbon number of at least 34.

79. The formulation of claim 77, said liquid wax ester including jojoba oil.

80. The formulation of claim 1, said hydrophilic clay predominantly including at least one smectite.

81. The formulation of claim 80, said smectite selected from at least one of the group consisting of bentonite, montmorillonite, and hectorite.

82. The formulation of claim 80, said smectite including bentonite and said base including jojoba oil.

83. The formulation of claim 76, the formulation containing at least 3% or at least 5%, by weight, of said humectant.

84. The formulation of claim 77, the formulation containing, by weight, between 12% and 55% of said liquid wax ester.

85. The formulation of claim 71, the formulation containing, by weight, at least 0.005% of said silver(II) oxide.

86. The formulation of claim 71, the formulation containing, by weight, at least at least 0.10% of said silver(II) oxide

87. The formulation of claim 1, wherein said base includes water.

88. The formulation of claim 1, the formulation containing, by weight, at least about 1% of a skin-protecting agent.

89. The formulation of claim 80, the formulation containing said smectite and said silver(II) oxide in a weight ratio of up to 50:1 of said smectite to said silver(II) oxide.

90. The formulation of claim 89, wherein said weight ratio is at least 0.2:1.

91. An antibiotic formulation suitable for application to skin tissue, the formulation comprising:
(a) a silver(II) oxide;
(b) a hydrophilic clay; and
(c) a base,
said hydrophilic clay predominantly including at least one smectite in a weight ratio of 0.2:1 to 50:1 of said smectite to said silver(II) oxide,
said silver(II) oxide and said smectite being intimately dispersed within said base.
